(19) United States
(12) Patent Application Publication
(10) Pub. No.: US 2018/0340927 A1
(43) Pub. Date: Nov. 29, 2018

(54) SYSTEMS AND METHODS FOR MONITORING AN INDIVIDUAL'S HEALTH

(71) Applicant: Wellmetris, LLC, Keego Harbor, MI (US)

(72) Inventor: Andrew A. Dahl, Bloomfield Hills, MI (US)

(21) Appl. No.: 16/054,723

(22) Filed: Aug. 3, 2018

Related U.S. Application Data
(63) Continuation-in-part of application No. 13/812,220, filed on Jan. 25, 2013.
(60) Provisional application No. 61/367,486, filed on Jul. 26, 2010.

(51) Int. Cl. G01N 33/487; G01N 33/493; G01N 33/52; G01N 33/70; G16H 10/40; G16H 50/70; G16H 50/30; H04L 29/08; G08C 17/02

(52) U.S. Cl. CPC G01N 33/48792; G01N 33/493; G01N 33/52; G01N 33/70; G08C 2200/00; G16H 50/70; G16H 50/30; H04L 67/125; G08C 17/02; G16H 10/40

(57) ABSTRACT

In various embodiments, a metabolic urinary test core platform is disclosed. The core platform is adaptable into systems and methods for monitoring an individual's health. The core platform comprises a model; a physiologic fluid; a test panel; an instrument; and a computer, the computer further comprising program instructions written on a non-transient computer-readable media for performing a method of monitoring an individual's health. In certain aspects of the disclosure, the model comprises human individuals categorized into groups based on age and/or lifestyle, such as normal adult, workplace worker, elderly, athlete and dieter.

### CROSS-REFERENCE TO RELATED APPLICATIONS

This application is a continuation-in-part of U.S. application Ser. No. 13/812,220 filed Jan. 25, 2013 and entitled “WELLNESS PANEL.” The '220 Application is the U.S. national stage entry of International Application Serial No. PCT/US2011/044786, filed Jul. 21, 2011, which claims priority to U.S. Provisional Application Ser. No. 61/367,486 filed Jul. 26, 2010. Each of these disclosures is incorporated herein by reference in their entirety for all purposes.

### BACKGROUND

The present invention relates to human healthcare, and in particular, to systems and methods for individuals to use to assess and monitor their own health.

Traditional healthcare tests are designed and employed to diagnose specific diseases, with an increasing emphasis in recent years on early diagnosis of diseases. Traditional tests are generally not useful for the assessment of a person's wellness or relative health, or for the assessment of the risk in developing certain diseases. Some traditional tests analyze for certain physiological substances, such as cholesterol, lipoproteins, CRP (c-reactive protein), albumin/creatinine ratio, and other “risk factors” that may be indicative of specific diseases, e.g. cardiovascular disease. However, the disease-specific application of these pre-symptomatic medical tests is consistent with traditional medicine's focus only on the diagnosis of specific diseases, and most often cardiovascular disease.

For example, although chronic inflammation is associated with a significant increase in the risk for certain cancers, and regular use of drugs or dietary agents with anti-inflammatory activity have been proven to reduce the risk for such cancers, traditional clinical laboratories and clinicians do not monitor biomarkers for inflammation as risk factors for cancer. As a specific example, the currently available CRP test only interprets the level of CRP as a marker for cardiovascular risk.

With the exception of disease-specific application of these few medical testing examples discussed, at present none are readily available to individuals seeking to determine how healthy they are. Further, no testing is readily available to an individual for the individual to easily assess their own health based on a determination of inflammation levels, oxidative stress levels, antioxidant activity levels and/or other indicators that are health related. The methods cited above typically require complex instrumentation and technically skilled operators, making them expensive and not suitable for widespread application by ordinary persons. Further, many healthcare tests require samples be transported to specialized locations capable of performing such analyses.

Therefore, there still remains a need for new systems and methods for monitoring an individual's health comprising tests that ordinary individuals without medical training can perform at home to quantify analytes in their urine relating to important physiological conditions and their personal health. Further, there is a need for improved health monitoring systems that assess an individual's health and relative resistance to multiple diseases, which can be performed non-invasively at low cost, and which can provide accurate results regarding the health of the individual.

### SUMMARY

In various embodiments, a metabolic urine test core platform is disclosed. The core platform is adaptable into systems and methods that provide health monitoring of individuals classified into particular groups. In various embodiments, a metabolic urine test core platform comprises a model, a test panel, a physiologic fluid, a test panel, an instrument, and a computer further comprising program instructions written on a non-transient computer-readable media. In various examples, the model comprises human individuals, and specifically individuals categorized into certain groups. In various aspects, the physiologic fluid comprises urine, and correspondingly, the test panel comprises a urinary test strip. The program instructions may comprise algorithms, analytics, and a graphical user interface (GUI) to perform a method of health monitoring.

In various embodiments, systems and methods for monitoring an individual's health are disclosed. The systems and methods are adaptations of the metabolic urine test core platform and are optimized for certain groups of individuals. Groups that individuals may be classified into include, but are not limited to, a normal adult, a workplace worker, an elderly person, an athlete, and a dieter. As described in detail herein, each of these groups require uniquely customized health monitoring systems and methods because of the differences in how the presence, absence or levels of biomarkers relate to an individual's health across these groups.

In various embodiments, health monitoring systems and methods comprise test panels for monitoring levels of various biomarkers in an individual, such as biomarkers indicative of inflammation, oxidative stress, antioxidant activity, ketoacidosis, protein toxicity, and so forth. In certain aspects, test panels are structurally designed as urinary test strips to be wetted with an individual's urine sample. In various embodiments the selection of the combination of particular assays present on a test strip is critical to the group identity. For example, a test strip for an elderly individual to monitor their health is structurally different in the combination of assays present from a test strip for an athlete to monitor their health, and so forth.

In various embodiments, a system for monitoring an individual's health comprises a test panel, such as a urinary test strip further comprising biomarker assays, an assay reading device, and a computer further comprising program instructions written on a non-transient computer-readable media for performing the method. In certain aspects, the computer comprises a portable electronic device such as a smartphone. The assay reading device may be dimensionally small, optionally small enough to be hand held, wherein the device is in communication or otherwise electronically connected with a computer such as a smartphone for the transfer of data from the device to the non-transient computer-readable media of the smartphone.

In various aspects, a method of individual health monitoring comprises collecting a urine sample from an individual desirous of a health assessment or continued health monitoring, wetting a test panel with the urine sample, inserting the test panel into an assay reading device, acquiring data from the urine sample, transferring data from the device to the non-transient computer-readable media comprising program instructions, analyzing the data including normalization and indexing, optionally reporting the data to the individual on a screen or on a printout, and optionally uploading the data to the Cloud or a remote server for meta-analysis. In certain examples, a urine sample is applied to a test strip comprising a critical combination of biomarker assays, levels of various biomarkers are determined through performance of the assays, (e.g. biomarker levels relating to inflammation, oxidative stress, antioxidant activity, toxicity, etc. are determined by the various assays on the strip) and the individual's relative health and susceptibility to certain diseases is assessed from the data.

### DESCRIPTION OF THE DRAWINGS

Other advantages of the present invention are readily appreciated as the same becomes better understood by reference to the following detailed description when considered in connection with the accompanying drawings wherein:

FIG. 1 is a flow chart that begins with collection of a sample an ends with a result displayed on a user interface;

FIG. 2 is an example of a user interface showing an embodiment of a test results screen in which normalized biomarkers related to BMI are analyzed against various physiological conditions;

FIG. 3 is an example of a user interface showing an embodiment of a result in which oxidative stress is elevated;

FIG. 4 is an example of a user interface, which shows an embodiment of how chronic inflammation, oxidative stress, and various diseases may be linked;

FIG. 5 shows an embodiment of system components, which comprise a multianalyte assay carrier, a disposable sample collection cartridge, a wireless optical analyzer device, and a personal electronic device application;

FIG. 6 shows an embodiment of system components comprising a multianalyte assay carrier inserted into an analyzer, a wireless optical analyzer device, and a wellness panel results display.

### DETAILED DESCRIPTION

The present disclosure provides a metabolic urine test core platform adaptable for individual health monitoring based on urine sample testing and analysis of data. The core platform adapts to provide systems and methods for health monitoring that are customized to certain groups of individuals, such as normal adults, workplace workers, elderly, athletes, and dieters. In certain aspects, systems and methods for health monitoring comprise customized test panels in the form of urinary test strips for monitoring the levels of select biomarkers in human urine. Most generally, the test strips may include a set of chemical, immunochemical and/or enzymatic assays that are used together for monitoring the levels of a critical set of biomarkers. In some examples, test strips may monitor biomarkers indicative of inflammation, oxidative stress, and/or anti-oxidant activity in an individual. In other examples, such as for individuals on a paleo diet, test strips may also monitor biomarkers indicative of toxicity, such as urea, protein toxicity and ammonia.

### Definitions and Interpretations

The term “assay” as used herein refers to a chemical test that qualitatively identifies presence or absence of, or quantitatively or semi-quantitatively determines the amount of, a particular biomarker in a urine sample. An assay herein may comprise a chemical, immunochemical or enzymatic assay based on reagent chemistry, enzyme action or antibody/antigen bonding. The assay may comprise a dry pad located on a test strip, or a porous plastic strip that a urine sample can wick through. The biomarker detected and/or measured may be indicative of a medical condition.

The term “biomarker” as used herein refers to a physiological substance present in the urine of a human individual that is measurable or at least detectable, such as, but not limited to, a ketone, ammonia (NH₃), a protein, an organic acid, an amino acid, a nucleotide derivative, a metabolite, various oxidation products of physiological substances, a DNA or RNA oligomer, or other biological substance, normally or abnormally present. Levels of biomarkers, or just their presence or absence in an individual, may relate to an individual's general health. Some assays are designed to detect/measure individual biomarkers whereas other assays may detect/measure groups of biomarkers, based on the chemistry and the sensitivity of certain assays to various biomarkers.

The terms “healthy” or “in good health” as used herein refer to a subjective state of a person who is generally free from detectable disease and who has a relatively low risk of developing certain diseases. Healthy may also be associated with the ability for the individual to engage in daily activities without discomfort, and in some instances, may be associated with the ability for the individual to pass a stress test and a physical exam with no issues. The assessment of “healthy” for an individual may be relative to an average population of similar individuals, or relative to an earlier assessment of the same individual. Healthy may also be a conclusion as to the state of an individual when the level of a particular biomarker in an individual is determined to fall within acceptable limits for similar individuals in that same age group and lifestyle.

As used herein, the term “health assessment” refers to qualitative or quantitative information relating to an individual's state of wellness. A health assessment may be relative, in that any single health assessment determined on a particular day may be compared to any number of previously obtained health assessments in order to track a trend in an individual's health, wherein the trending may be more important than any individual determination. The units of measurement of a health assessment may be entirely qualitative, such as “good” or bad.” In other embodiments, a health assessment may be a quantitative result of a particular biomarker having scientific units of measurement, such as μg/dL or normalized as ng/mg creatinine. For example, a health assessment may be the normalized level of a particular biomarker in an individual's urine, such as the level of malondialdehyde (MDA), 8-hydroxy-2′-deoxyguanosine (8-OHdG), or other biomarker in the individual's urine at a particular point in time, normalized to creatinine. An individual may be able to recognize on their own whether or not a particular normalized level of a biomarker in the urine relates to a “good” or “bad” health assessment, or the individual may rely on computer software or a chart of normal biomarker ranges to make the inference. In various embodiments, a health assessment may comprise quantitative measures of inflammation biomarkers, antioxidant capacity biomarkers, oxidative stress biomarkers, and/or toxicity biomarkers, singly or in various combinations. In certain aspects, a health assessment may be in the form of an additional data point on a plot of biomarker values taken over time for an individual, such that the individual can see trends in that biomarker over time compared to a normal range (minimum to maximum) for that biomarker.

The term “sample” as used herein refers to a urine sample from a human individual. It should be noted that certain biomarkers can be present in one type of physiologic fluid but not in others, and that a biomarker measured may be specific only to urine or more widespread across various bodily fluids. Herein, there may be use of the phrase “collecting a urine sample from an individual.” This phrase is not intended to imply the intervention of a medical professional to “collect the sample.” On the contrary, the tests herein are designed to be conducted at home by the individual. Therefore, the individual supplies their own urine sample to test, such as by urinating into a cup or urinating directly onto an end of a test strip at home. In some instances, a test may be conducted by a medical professional, such as if the individual needs training in the use of a test.

The term “test panel,” (or more simply, “panel”), as used herein, refers to a test strip further comprising a critical combination of assays. In certain aspects, a “test panel” comprises a dipstick-style test strip wherein the assays may be arranged in a straight line as dry reagent pads along the length of the strip, separated with enough space between them such that the assays don't interfere with one another (such as bleed color from one to the next).

The term “dry chemistry” as used herein refers to an assay comprising dried or absorbed chemical reagents that are dissolved, mobilized, and/or reacted once the assay is wetted with a urine sample. The simplest common example of a dry chemistry assay is a strip of litmus paper. A more complicated example is a barcode-style lateral flow immunoassay wherein antibodies and/or enzymes and other reagents begin absorbed into a porous plastic strip at certain positions, the familiar example being a home pregnancy test. In these and other examples of dry chemistry assays, the assay appears “dry” to the naked eye, in contrast to having a vial of liquid reagents in a clinical laboratory into which a fluid sample like urine may be added.

As used herein, the term “normalization” or “standardization” refers to the correction of a biomarker value for variations in hydration and urinary output of an individual. Since the amount of creatinine excreted daily in the urine by an individual is relatively constant, urinary creatinine levels are often used to correct the levels of other urinary biomarkers. So for example, an uncorrected level of a biomarker may be expressed in units of ng biomarker/dL, which may not be that meaningful prior to normalization. When normalized by the urinary creatinine level obtained from the same urine sample, the normalized biomarker measurement may be expressed in units of ng biomarker/mg creatinine, where the biomarker/creatinine ratio was obtained simply by mathematical division of the two. Other normalization mechanisms not dependent on creatinine assays may be necessary if creatinine is at abnormal levels in an individual, such as an athlete engaging in daily anaerobic exercise or marathon running.

As used herein, the term “indexed” or “indexing” refers to a comparison (by definition, an indexing) of a normalized biomarker level to a chart of normal ranges for individuals of a particular age and gender, and if female, whether pregnant or not. Thus, indexing a previously normalized biomarker measurement means to compare the measured and normalized level of the biomarker to a chart of normal ranges of that biomarker for certain individuals. Herein, a urinary biomarker is both normalized and indexed so that it provides an indication of an individual's relative health. In some instances, a chart of normal ranges for a biomarker may be referred to as a “lookup chart” for that biomarker. An individual may be able to see a lookup chart, such as on a piece of paper, or the indexing charts may be incorporated into algorithms that assess an individual's health given the normalized biomarker data and facts about the individual (age, lifestyle, the group classified in, etc.).

As used herein, the term “PED” takes on the common meaning of a portable electronic device, such as, for example, smartphones, tablets, e-readers, laptop computers, and so forth. Although “smartphone” may be recited as an exemplary device for use herein, the implication is that any other portable device may be used instead. For example, where it is recited that an app may be downloaded to a smartphone, the app could of course be downloaded and used on a tablet, laptop computer or any other processor-based device. For the systems herein, a “computer” is referred to. The understanding is the computer may comprise a PED.

As used herein, the term “app” takes on the common meaning of a small, specialized software program that can be downloaded onto non-transient computer-readable media within a PED such as a smartphone. In various embodiments, an app is downloaded onto a smartphone and used by an individual to obtain his/her health assessment and health monitoring based upon the results obtained from a test strip of assays, along with added personal data, (age, gender, etc.). As typical for an app, certain information may be first entered and then stored by the app such that the user does not need to keep entering the same information each time the app is used. In various aspects, an app herein normalizes a biomarker measurement (such as by dividing two measurements) and then indexes the normalized level with a chart of normal ranges for that biomarker for specific individuals (age, gender, etc.) in a predetermined group of individuals. An app may further include program instructions to upload biomarker and/or health assessments to the Cloud or to a remote server, such as at an employer, a healthcare provider or an insurer.

The term “model” as used herein refers to “who” the metabolic urine test core platform is customized for. In various embodiments, the model comprises human, and more specifically, the model comprises human individuals classified into certain groups for further customization of the health monitoring systems and methods.

The term “groups of individuals” as used herein refers to lifestyle and/or age-related classes of individuals, segregated for the purpose of obtaining customized, appropriate health monitoring systems and methods. It should be noted that the group classification may be objective or subjective, and it is possible for an individual to find themselves classifiable into more than one group. An individual may believe they belong in a particular group, and insist so, or they may be guided to such classifications by a medical professional or anyone else. For example, a marathon runner who happens to be over 65 years old may believe they should be classified into both “elderly” and “athlete” groups so that they can benefit from the health monitoring systems and methods customized for each of these two groups. In another non-limiting example, a person on a calorie-conscious diet may not see themselves as being classified in the “dieters” group because they feel that they are not stressing their bodies to the extent that perhaps a binge-dieter may be, or a paleo-dieter who also happens to be Type-I diabetic and at risk for protein poisoning, ketoacidosis and organ failure because of the dieting. A person who drastically changes their diet may want to classify him/herself as a “dieter” to get the appropriate health monitoring system and method.

The term “normal adult” refers to the typical adult ranging in age from about 18 to about 65 years old, who may have a daily job and family life, but eats ordinary food and exercises perhaps regularly or very little at all. For example, the normal adult may be an office worker, a housewife, or a college student. Although “ordinary” is a subjective term, a “normal adult” herein refers to the majority of young and middle aged individuals who are just living an ordinary life without any physical stresses, binges, or voluntary excesses, like engaging in extreme physical training or extreme dieting. A normal adult may be somewhat out of shape (a less than ideal body mass index BMI and weight, for example), but wouldn't be viewed by the medical profession as being obese or anorexic.

The term “workplace worker” refers to the normal adult who has a physically demanding work schedule that may impinge on their personal health, or who is employed by an employer who suggests or mandates employee health monitoring for insurance or other reasons. Although subjective, the workplace worker may be a factory worker, or agricultural worker who may be exposed to toxins, or workers in physically demanding jobs. For example, the workplace worker may be a roofer who works in extreme climates, or an exterminator desirous of monitoring themselves for signs of deteriorating health. In various examples, the workplace worker may also be an office worker who is mostly or entirely sedentary. Such a person may be concerned about developing cardiovascular disease from their sedentary lifestyle, and may desire specialized health monitoring as a “workplace worker” so that health changes relating to the onset of cardiovascular disease may be detected. In various regards, classification as a workplace worker allows for workplace wellness pre-clinical screening, and the systems and methods of health monitoring in the workplace may also be used by employers and insurers.

The term “elderly” as used herein refers to individuals greater than about 65 years of age. Most of the developed world considers this age as the start of a person's elder years.

The term “athlete” as used herein refers to individuals that strenuously exercise at least three times per week, engaging in such strenuous exercising like weightlifting/body building or marathon running, or other rigorous and regular training regiment. Athlete generally refers to individuals that exercise to such an extent that they risk health issues such as dehydration and organ damage. Athlete herein includes professional athletes that have to train constantly for their professions. Bodybuilders may overindulge in protein consumption, risking their metabolic health. Marathon runners often run several miles each day to remain competitive, risking organ stress, elevated creatinine levels and other issues. Competitive tennis players may train for hours each day in the heat. However, an individual who merely steps on a treadmill at home three days a week may not want to classify him/herself as an “athlete,” but instead, “normal adult.”

The term “dieter” refers to individuals engaging in an extreme diet, or at least changing their diet significantly or radically from what it was. Although subjective, the classification of dieter is meant to include persons that, for one reason or another, change their diet fairly radially. Non-limiting examples may be a person who once ate significant amounts of carbohydrates daily (lots of bread, cereal, pasta) and then decides to follow a strict protein-rich Atkins Diet. Another example of a dramatic shift in diet may be that same carbohydrate-loving person who shifts to a Paleo Diet. In some instances, an individual who already has a preexisting health condition, such as a diabetic, may want to classify him/herself as a “dieter” when making even small changes in their diet. For example, a Type-I diabetic who normally eats balanced levels of carbohydrates and proteins and takes insulin injections regularly in respond to blood sugar levels, may want to be classified as a “dieter” if shifting their diet to a Paleo Diet that is almost devoid of carbohydrates. In this way, “dieters” benefit from customized systems and methods of health monitoring because their extreme change in eating, perhaps coupled with a preexisting condition such as diabetes, may lead to protein toxicity or abnormal levels of NH₃ that present serious health concerns.

### The Metabolic Urine Test Core Platform

In various embodiments, a metabolic urine test core platform comprises (a) a model; (b) a test panel; (c) a physiologic fluid; (d) a test panel; (e) an instrument; and (f) a computer further comprising program instructions written on a non-transient computer-readable media. Adaptations of this core platform, i.e., through various selections of (a)-(f), provide systems and methods for monitoring an individual's health. In other words, the core platform is flexible. For purposes herein, the core platform addresses a human model, and in particular, a model comprising human individuals classifiable into various groups based on age and various lifestyle factors. For purposes herein, the test panel comprises a dry chemistry test strip, such as a dipstick, which further comprises chemical, enzymatic or immunochemical assays in the form of dry reagent test pads and/or barcode-style lateral flow immunoassays. In various embodiments, the instrument may comprise a portable or handheld assay reading device into which a wetted and reacted test strip may be inserted, wherein the device may further comprise colorimetric capabilities to take reflectance or transmission readings off or thru the test pads and/or off or thru the lateral flow immunoassays. In some embodiments, the instrument may be just the human eye, whereby an individual counts darkened or colored bars or assesses a color change or development of color in an assay without the need for actual instrumentation. The algorithm and analytics present in the written instructions obtain and interpret data from the instrument, and normalize and index the data, as explained below. The computer may comprise a PED, like a smartphone, which the individual can use to obtain their health assessment and track their own health over time. In various embodiments, the computer comprises a PED having software written on a non-transient computer-readable medium for performing a method of health monitoring.

The metabolic urine test core platform disclosed herein has been designed to overcome limitations of using tests in home settings or large-scale, pre-clinical screening efforts. When adapted to specific groups of individuals, the systems and methods for health monitoring herein detect early changes in broad metabolic measures before blood tests such as HBA1c, would indicate a specific abnormal result, effectively functioning as an early assessment and not a diagnostic. The non-invasive nature of the test panels, configured for use with urine samples, may encourage more compliance among voluntary participants, as opposed to blood draws or finger sticks for workplace wellness screening programs and elsewhere. In the systems and methods herein, data are processed to display results at an informational level on-site and in real-time on a smartphone, tablet or laptop. Census data is also available to the test sponsor and/or insurer. For workplace wellness use, the systems and methods adapted herein for the workplace worker capture data and create a HIPAA-compliant record, automating health risk assessments. Further, the systems and methods are compatible with most medical records applications, thus minimizing clerical costs associated with charting test results.

For at-home personal use, the systems and methods herein allow individuals to monitor the benefits of their diet, exercise and supplementation regimen by providing information in the form of oxidative stress, inflammation, hydration and ketone levels through an easy to interpret algorithm and attractive graphics. For the health-conscious, this represents a new level of engagement in their quest for optimal health and longevity.

The systems and methods disclosed herein represent a significant departure from traditional clinical diagnosis, which seeks to diagnose diseases. The focus of the systems and methods herein is to assess, non-invasively, how healthy an individual is by monitoring various combinations of biomarkers relevant to the health of individuals in certain groups of individuals. In various embodiments, three factors may be monitored, two of which are directly related to risk of disease (oxidative damage and inflammation) and one (antioxidant activity) which is inversely related to the risks of chronic diseases such as cancer, CVD, neurodegeneration, among others. A test panel comprised of assays for one or more urinary biomarkers relevant to all three of these factors has not been previously disclosed, nor has a test panel comprised of assays for biomarkers for these conditions been combined previously with other information, such as body mass index calculations and/or an individual's lifestyle.

Oxidative stress and chronic inflammation have been extensively investigated and widely accepted as major, underlying factors in a range of human pathology that includes cardiovascular disease, diabetes, stroke, and cancer. Awareness of the effects that lifestyle choices may be having on disease development can help mitigate pre-conditions favorable to the onset of disease and impairment. The core platform is adapted to systems and methods of health monitoring that further comprise simple, inexpensive, pre-clinical screening tests to detect the oxidation/inflammation process at both the earliest and later stages to help individuals make healthy lifestyle choices, and from a prevention perspective, to allow providers and insurers to better gauge health risk within a largely asymptomatic group, such as employees, retiree pools, or government dependents.

### Collecting and Analyzing a Sample

A sample is obtained from a user. In embodiments, the sample is blood sample. In embodiments, the sample is a urine sample. The sample may be obtained through any method known in the art. In embodiments, the sample is obtained using a wick.

The sample is then assayed to determine the concentration of certain analytes. In embodiments, the number of analytes measured is between 1 and 10. In embodiments, the number of analytes measured is between 10 and 50. In embodiments, the number of analytes measured is between 50 and 100. In embodiments, the number of analytes measured is greater than 100.

In embodiments, the analytes being measured comprise any chemical substance. In embodiments, the analytes being measures are proteins.

Any assay known in the art may be used to determine the concentration of the analytes. In embodiments, the assay used is UV/Vis spectroscopy. In embodiments, the assay is a colorimetric assay.

In embodiments, the concentration of the analytes is then communicated to a wireless device. In embodiments, the wireless device then converts the concentration of the analytes to a different value. In embodiments, the concentration of the analytes is converted to molarity. In embodiments, the concentration of the analytes is converted to molality.

The molar values are then normalized as they relate to certain biomarker ranges. In certain embodiments, the biomarker ranges are considered normal values for the user's age. In other embodiments, the biomarker ranges are considered normal for the user's gender. In yet other embodiments, the biomarker ranges are considered normal for the user's body mass index. Alternatively, in other embodiments, the biomarker ranges are considered normal for a person who is pregnant. In yet other embodiments, the biomarker ranges are considered normal for a person who is not pregnant. In still yet other embodiments, the biomarker ranges are considered normal for a person who has diabetes. Finally, in other embodiments, the biomarker ranges are considered normal for a person who does not have diabetes.

Based on normalizing the molar values to the biomarker ranges, a process algorithm is used to determine whether the user has any of the physiological conditions in a relational database. In embodiments, the relational database contains any physiological condition known in the art. In embodiments, the relational database contains any one or more the following physiological conditions: oxidative stress, systemic inflammation, total antioxidant capacity, autoimmune diseases, cardiovascular diseases, cancer, and diabetes.

The results are displayed on a user interface and can be stored in a cloud.

### Biomarkers Measured by the Test Strip Assays

The study of metabolites in bodily fluids and excretions is creating new momentum behind a biomedical backwater, namely dry-chemistry urinalysis. Unlike genomics, which offers practitioners, researchers, and actuaries not much more than a risk profile when applied to patient care or intervention, or proteomics, which confronts the researcher with thousands of proteins and complex interactions, the field of metabolomics, and specifically urine-based metabolomics, is focused on the several hundred metabolites that are passed into urine. These metabolites are, for the most part, relatively stable because they are essentially the waste products of healthy or unhealthy metabolic processes. They are proof positive that something has or is occurring in the individual and a type of footprint that can be measured, analyzed and monitored over time. Using clinical data, these metabolites can be correlated to metabolic efficiency and the general health of an individual or group of individuals. In the systems and methods herein, data from individual health monitoring can be uploaded for meta-analysis, such as an indexing with long established analyte ranges for particular age groups and gender. Establishing a baseline and monitoring the improvement or decline in metabolic status by tracking certain biomarker levels provides the motivation and documentation that intervention specialists, nutritionists, and wellness coaches can use to help shape healthy lifestyle choices.

As set forth in TABLE 1, urinary biomarkers may be classified as to what physiological condition they relate to, such as inflammation, oxidative stress, antioxidant capacity, toxicity, etc., or what other purpose their measurement may have, e.g. normalization of other biomarker measurements. Selection of a particular combination of biomarkers for a particular test strip depends on the identity of the group of individuals the health monitoring is customized for. As mentioned, a critical combination of biomarkers is selected for a group of individuals with certain age and lifestyle factors, because certain biomarkers are relevant to health monitoring of individuals in certain groups. Further selection criteria include the reliability, selectivity, and sensitivity of each biomarker assay, the stability of the biomarker (e.g. relatively low reactivity with air and/or light once the urine sample is exposed to air), relatively low reactivity with other components of the urine sample, such as reactivity with proteins to form adducts or the proteolytic degradation of protein biomarkers, and the ease of quantifying the biomarkers with a simply assay, such as colorimetric, without the need for sophisticated equipment (e.g. LC/MS).

TABLE 1
Biomarkers and Assays Relating to Health of an Individual
Wellness Biomarkers and Assays

| | Used as a | |
|---|---|---|
| | biomarker in | |
| | Blood | Urine |
| OXIDATIVE DAMAGE | | |
| Broad measures of damage | | |
| TBARS | x | x |
| Organic Hydroperoxides | x | x |
| Protein Carbonyls | x | x |
| Urinary Ketones | | x |
| Measure of damage to specific molecules | | |
| Lipids | | |
| Malonaldehyde | x | x |
| 4-hydroxynonenal | x | x |
| Lipid hydroperoxides | x | x |
| Isoprostanes (e.g. F₂-isoprostane (F₂-isoP)) | x | x |
| Linoleic acid oxidation products | x | x |
| Proteins | | |
| Protein carbonyls | x | x |
| 3-Nitrotyrosine | x | x |
| Nitrothiols | x | x |
| Up to 100 other oxidized amino acids | x | x |
| Nucleic acids | | |
| 8-hydroxy-2′-deoxyguanosine (8-OHdG) | x | x |
| 8-hydroxyguanosine | x | x |
| M1dG | x | x |
| Oxidized derivatives of ribose ring | x | x |
| Small molecules and ions | | |
| Selenium | x | x |
| GSH or GSSG and the GSH/GSSG ratio | x | x |

ANTIOXIDANT POWER
Used as a biomarker in blood or urine:
Direct methods (measure reaction with a redox probe)
CUPRAC (cupric reducing antioxidant capacity)
Total Antioxidant Capacity (copper-bathocuprione method)
Indirect methods (measure resistance to oxidation of a probe by an
added oxidizer)
FRAP (ferric reducing ability of plasma)
TRAP (total reactive antioxidant potential)
ORAC (oxygen radical absorbance capacity)
HORAC (hydroxyl radical antioxidant capacity)
Measurement of molecules that contribute to the total antioxidant capacity
GSH or GSSG and the GSH/GSSG ratio
Glutathione Peroxidase
Superoxide Dismutase
Uric acid
Ascorbic acid

| | | |
|---|---|---|
| INFLAMMATION | | |
| Cytokines | | |
| TNF-α | x | — |
| IL-6 | x | x |
| IL-8 | x | x |
| Interleukin 1 family, Interleukin 2 family | x | x |
| Other proteins | | |
| Osteopontin | x | x |
| Orosomucoid | — | x |
| Albumin | — | x |
| α1-microglobulin | — | x |
| Immunoglobulins | x | x |
| Eicosanoids | | |
| PGE2 and metabolites | x | x |
| PGF2a and metabolites | x | x |
| Other molecules | | |
| Nitric oxide byproducts (NOx)(nitrate + nitrite) | x | x |
| Urinary Trypsin Inhibitor (also called bikunin or | | x |
| ulinastatin) | | |
| Neopterin (as an indicator of immune activation and | | x |
| CAD) | | |
| Urinary proteins | | x |
| Histamine | x | x |
| TOXICITY | | |
| Ketones (as a biomarker for ketoacidosis) | | x |
| Ammonia NH₃ (as a biomarker for protein toxicity) | x | x |
| Heavy metals (Hg, Pb, Cd, Cr, and other metal toxins) | x | x |
| Urea (in a ratio to nitric oxide byproducts) | | x |
| NORMALIZATION | | |
| Urinary Creatinine | | x |
| ENERGY & METABOLISM | | |
| Nitrogen/Urea Ratio (energy consumption in muscles) | | x |
| β-Hydroxybutyrate | | x |
| PUFA byproducts | | x |

In various embodiments, biomarkers for any individual test panel comprise substances that can be quantified quickly by chemical, immunochemical, and/or enzymatic assays that can be incorporated as dry chemistry assays on test strips and subsequently read using a compact and simple reflectance instrument or even visually once the assays are exposed to a urine sample. In certain aspects, one or more of the biomarkers selected for inclusion in a test panel may require the use of antibodies, including lateral flow immunoassays that may be read visually or by various colorimetric, radiometric, fluorometric or chemiluminescent methods. In certain example, one method in a single device may be employed to detect and analyze biomarkers in all three categories of inflammation, oxidative stress and antioxidant capacity. However, each biomarker assay can also comprise a different method. For example, one biomarker can be analyzed by an immunoassay, and another biomarker can be analyzed by a chemical indicator e.g. a color change. When on a single device, preferably the assays are physically separate, such as having test pads on a hydrophobic backing dipstick material with the option of blotting excess urine from the dipstick for minimal crosstalk between test pads. Any single test strip of biomarker assays will necessarily include a normalization assay, such as a urinary creatinine assay in order to normalize for variations in urine concentration. The results of the combination of assays for a particular individual in a group may then be compiled into a report on the individual's health based on peer-reviewed standards for indexing. The report may be viewed, for example, on the individual's smartphone.

The test strip, comprising a critical combination of biomarker assays and performed on urine specimens, provides a more robust assessment of an individual's health status than from any one assay alone. In various examples, a test panel comprises at least one biomarker assay informative of each of inflammation, oxidative stress, and anti-oxidant activity, adapted into a simple dipstick test strip structure employing dried reagents, or reagents impregnated into a lateral flow immunoassay device. In various examples, a test panel adapted into a simple dipstick test strip further comprises at least one normalization assay, such as a urinary creatinine assay. In certain examples, a test strip comprises a normalization assay and at least one other urinary biomarker assay, positioned on the same strip as dry chemistry assays.

### Oxidative Stress

Oxidative damage in the tissues and cells of an individual may be the result of reactive oxygen species (ROS), such as peroxides and oxygen radicals. Some level of ROS is normal in an organism and certain ROS species take part in normal biochemical pathways. However, excessive ROS levels cause oxidation of certain biomolecules in an individual, and the oxidation products, or derivatives therefrom, may appear in bodily fluids such as blood or urine. ROS can be produced from fungal or viral infection, ageing, UV radiation, pollution, excessive alcohol consumption, and cigarette smoking among other diseases. ROS can further cause age-related macular degeneration and cataracts. Of primary interest are the oxidation products of certain fatty acids and DNA, as the appearance of the oxidation products from fatty acids or DNA can be indicative of excessive ROS and the existence of oxidative damage at the cellular level. Oxidation of fatty acids in an organism is often referred to as “lipid peroxidation.” Further, ROS also includes the reactive nitrogen species (RNS), which includes nitric oxide radical ⋅NO and ONO₂—. These reactive species cause “nitrative stress,” with RNS reaction products including such molecules as 3-nitrotyrosine. Since the RNS species are in effect ROS species, nitrative stress is normally lumped together with oxidative stress when referring to oxidative damage in individuals.

In lipid peroxidation, it is the unsaturated fats that are most prone to oxidation, particularly arachidonic acid and linoleic acid with their polyunsaturated carbon chains. For example, oxidation of arachidonic acid and linoleic acid produces malondialdehyde (MDA) and 4-hydroxynonenal (4-HNE), amongst other products, which are secreted in the urine. MDA and 4-HNE can be measured in a urine sample as oxidative stress biomarkers.

An oxidative stress assay may comprise a specific malondialdehyde (MDA) or 4-hydroxyonenal (4HNE) method to quantify lipid peroxidation and/or a thiobarbituric acid reactive substances (TBARS) method to measure a broader range of substances oxidized to aldehydes and ketones due to the actions of free radicals. A ferrous reaction reagent suitable for use in assaying oxidative stress comprises 2-deoxyglucose, TBA, EDTA, and ferrous sulfate. Oxidized derivatives of amino acids in proteins are also biomarkers of oxidative stress. In principle, an oxidative stress biomarker can be any amino acid that has undergone oxidation or some other modification. For example, dityrosine and 3-nitrotyrosine are oxidative stress biomarkers produced by the reaction of tyrosine with peroxynitrite, or chloro-tyrosine, which is produced by the action of myeloperoxidase and is an inflammatory biomarker. Urinary 3-nitrotyrosine excretion is a urinary biomarker that reflects excessive ROS in an individual, such as ONO₂—. 3-Nitrotyrosine is the major product of tyrosine oxidation, although it is not clear if tyrosine is oxidized when in free form or when part of a polypeptide. See, for example, Radi, R., “Nitric oxide, oxidants, and protein tyrosine nitration,” Proc. Natl, Acad. Sci., 101, 4003-4008 (2004). Further, oxidized sulfur- or selenium-containing amino acids (collectively referred to as “SSAA”) are oxidative stress biomarkers. Oxidized SSAA are amino acids in which the sulfur or selenium moiety has been oxidized to a higher oxidation state. Oxidized SSAA include, but are not limited to, cysteine, cystine, methionine, selenomethionine, selenocystine and selenocysteine in their various possible oxidation states. In general, high levels of any one of these biomarkers indicate that oxidative stress is occurring in an individual. Low levels of these biomarkers indicate a healthy individual. Examples of ranges are given in the FIGURES for both oxidative damage and oxidative stress calculated from oxidative damage and total antioxidant power.

Additional oxidation and nitration products of lipids, proteins and DNA that find use as oxidative stress biomarkers include isoprostanes, 8-hydroxyguanosine and 8-hydroxy-2′deoxyguanosine. Oxidative damage to DNA can be evidenced by oxidation products of the most susceptible base, guanosine. The oxidation products that can be found at elevated levels in urine when excessive ROS are present include 8-hydroxyguanosine and 8-hydroxy-2′-guanosine. These substances have been shown to be useful biomarkers of oxidative stress. See, for example, Shigenaga, M. K., et al., “Urinary 8-hydroxy-2′deoxyguanosine as a biological marker of in vivo oxidative DNA damage,” Proc. Natl. Acad. Sci., 86, 9697-9701 (1989).

Isoprostanes found in urine primarily consist of 8-iso-prostaglandin F_{2α}, referred to more simply herein as F2-isoprostane, or F2-isoP. F2-isoPs are chemically stable prostaglandin-like isomers, generated by the reaction of polyunsaturated fatty acids and ROS, and have been shown to be useful biomarkers for oxidative stress in an individual. See, for example, Cracowski, J.-L., et al., “Isoprostanes as a biomarker of lipid peroxidation in humans: physiology, pharmacology and clinical implications,” Trends Pharmacol. Sci., 23, 360-366 (2002)).

Glutathione (GSH) is a tripeptide molecule that acts as an antioxidant, reducing various ROS species to become oxidized to the disulfide, GSSG. Since both the oxidized (GSH) and reduced (GSSG) species exist naturally, what is more important for health assessment is the ratio of GSH/GSSG. This ratio is about 30-100 in cytosol of cells, and about 3-10 in serum. The ratio decreases in the presence of oxidative stress. That is, there is an abnormally low level of GSH, and abnormally high level of GSSG, or both, causing the GSH/GSSG ratio to be lower than normal. See, in general, Frijhoff, J., et al., “Clinical relevance of biomarkers of oxidative stress,” Antioxid. Redox Signal., 23(14), 1144-70 (2015).

Uric acid is a degradation product of purine, and is indicative of an inflammatory factor that increases oxidative stress and promotes activation of the renin angiotensin aldosterone system. Thus uric acid is a useful urinary biomarker indicative of oxidative stress and overall health.

N-hexanoyl lysise (HEL), or more simply, hexanoyl-lysine adduct, is another lipid peroxidation biomarker. It is the product of omega-6 polyunsaturated fatty acid oxidation and is therefore elevated levels of HEL are indicative of excessive ROS in an individual. HEL concentration in human urine has been reported to be 22.9 nmol/L. See Sakai, K., et al., “Determination of HEL (hexanoyl-lysine adduct): a novel biomarker for omega-6 PUFA oxidation,” Subcell Biochem., 77, 61-72, (2014).

### Antioxidant Capacity

In various embodiments, antioxidant capacity testing employs a CUPRAC (cupric reducing antioxidant capacity) method for measuring the sum of the antioxidant activity due to multiple species (uric acid, proteins, vitamins, dietary supplements) present in a urine sample (See e.g., Özyürek, M., Güçlü, K. and Apak, R., “The main and modified CUPRAC methods of antioxidant measurement,” Trends in Analytical Chemistry, 30: 652-664 (2011)). Alternatively, or additionally, modified methods can be used to specifically measure or to discriminate among uric acid, ascorbic proteins or other substances that contribute to the overall antioxidant capacity, thereby monitoring what is referred to as the “antioxidant reserve.” Several other biomarkers can be used to gauge antioxidant capacity and non-limiting examples are listed in TABLE 1 above. The CUPRAC method, and other methods that employ a redox indicator, directly measure the reaction of antioxidants with substances having the appropriate redox potential to effect a visible color change or a color interpretable by a simple colorimeter. A higher value for antioxidant power, that is, a greater level of biomarkers indicative of antioxidant capacity, indicates a healthy individual because the individual has compounds that can neutralize free radicals that cause oxidative damage and stress. Examples of ranges of antioxidant power are shown in the FIGURES.

### Inflammation

Inflammation is a normal, adaptive immune response that occurs in reaction to injury and infection. It can be triggered by pathogens, the effects of metabolic disorders (e.g., elevated blood sugar), cellular dysfunction, and oxidative stress. The same free radicals that lead to the cellular damage seen as a result of oxidative stress can initiate a pro-inflammatory signaling cascade that, if left unregulated, can result in chronic inflammation-induced cell apoptosis, malignancies, and metastases.

Inflammation is comprised of a complex series of physiological and pathological events, including the increased production of several proteins (e.g. cytokines such as IL-6 and IL-8, as well as COX-2 and the inducible form of nitric oxide synthase). The production of nitric oxide, by the inducible isoform of nitric oxide synthase can increase up to 1000 times during inflammation, and has been shown to be a useful biomarker for inflammation (See e.g., Stichtenoth, D., Fauler, J., Zeidler, H., Frolich, J. C., “Urinary nitrate excretion is increased in patients with rheumatoid arthritis and reduced by prednisolone,” Annals of the Rheumatic Diseases 54:820-824 (1995)). Because NO is relatively unstable, the production of NO can be tested by employing methods for the measurement of it degradation products nitrate and nitrite, i.e. measuring nitrite or the sum of nitrite and nitrate in a blood or urine sample, which are often abbreviated as NOx. Further, although very high levels of protein in urine are associated with kidney disease, it is known that the retention of blood proteins by the kidney is reduced by the effect of certain inflammatory cytokines, so that modest elevations in the levels of urinary proteins that are less than those associated with kidney disease can be used as a biomarker for inflammation. Several other biomarkers can be used to test for inflammation and non-limiting examples are listed in TABLE 1 above. Higher levels of inflammation biomarkers indicate that inflammation is occurring in an individual, possibly indicative of disease. Lower levels of inflammation biomarkers indicate a healthy individual. Examples of ranges of inflammation biomarkers are shown in the FIGURES. Chronic inflammation can lead to hay fever, atherosclerosis, and rheumatoid arthritis. Anti-inflammatory agents have also been shown to significantly reduce the incidence of heart disease, diabetes, Alzheimer's disease, and cancer.

### Toxicity

In various embodiments, the systems and methods for monitoring an individual's health may comprise toxicity monitoring by including at least one biomarker assay relating to toxicity. The nature of human toxicity depends on lifestyle. For example, a metallurgist may develop heavy metal toxicity over time, whereas an elite athlete, such as a marathon runner, may develop, such as from time to time, ammonia and/or protein toxicity. Various other substances may be monitored by incorporating the appropriate assay on a customized urinary test strip. These include, but are not limited to, albumin, non-esterified fatty acids (NEFA), β-hydroxybutyrate, acetoacetate, creatinine, kidney injury molecule-1 (KIM-1), neutrophil gelatinase-associated lipocalin (NGAL), urinary proteins including clusterin, a glycoprotein, renal papillary antigen-1 (RPA-1), glutathione S-transferase-yb1 (GST-Yb1), glutathione s-transferase (α-GST), cystatin C, osteopontin, ammonia (NH₃), mercury (Hg), lead (Pb), cadmium (Cd), and chromium (Cr).

### Relationship of Oxidative Stress, Antioxidant Capacity and Inflammation to General Health

There is a good deal of overlap in the underlying pathology and the manifesting symptoms of diseases related to oxidative stress and inflammation. Measureable indicators of oxidative stress and inflammation are not markers of disease per se, as they typically represent a systemic environment, while most chronic conditions occur at the tissue level. So, while a given measure does not provide diagnostic information, they are indicative of the underlying processes, which themselves can contribute to the disease state. For example, malondialdehyde (MDA) and H₂O₂ are measurable indicators of oxidative stress. MDA is considered a marker of lipid peroxidation, a process that is implicated in several diseases. H₂O₂ is a metabolic byproduct of oxidative stress causes oxidative injury when there is dysregulation between the production of ROS and antioxidants. Urinary trypsin inhibitors and urinary proteins are measurable indicators of inflammation, which when chronic, contributes to the development of metabolic syndrome, diabetes, kidney disease, and cardiovascular disease.

Urinary protein levels confer the strongest risk of cardiovascular death. Urinary proteins indicate a threefold risk for coronary heart disease and stroke and, in subjects with metabolic syndrome, a marked increase in cardiovascular mortality.

Urinary trypsin inhibitors are predicted of vascular inflammation, which damages the endothelial and epithelial layers and promotes vascular disease. Urinary trypsin inhibitor is a biomarker for chronic inflammation in Type-I and Type-II diabetes. Levels of urinary trypsin inhibitor in the urine correlate with blood CRP, which is considered an inflammatory predictor for atherosclerosis.

Significantly higher than normal levels of urinary ketones are associated with increasing heart failure severity.

In the ten years since the sequencing of the human genome, it has become increasingly apparent that, while genetics plays a major role in the development of diseases for a small percentage of the population, the overall impact of genetics on major non-infectious diseases in humans is only about 15-20%. Much more important, especially for the development of the diseases that account for most morbidity and mortality in developed countries (chronic diseases such as cancer, cardiovascular diseases, neurodegenerative and autoimmune diseases) are the impact of diet, lifestyle (including exercise, smoking, alcohol use) and the environment. All of these factors influence an individual's health and they result in increases or decreases in inflammation and/or oxidative stress. Moreover, the oxidative stress can trigger some reactions that increase the level of inflammation.

The importance of oxidative stress to human health is evidenced by scientific publications and the numerous biomarkers that have been reported for oxidative damage, as well as the development of several tests for antioxidant activity and the widespread application of the ORAC test to measure the antioxidant activity in foods and juices, and the enormous market for nutraceutical supplements that have antioxidant activity in vitro. However, as has been now clearly demonstrated in the case of vitamin E, antioxidant activity in vitro does not necessarily translate into a change in the level of oxidative stress in vivo.

In keeping with traditional medical practices, some biomarkers for inflammation and oxidative damage have been translated individually into clinical practice. C-reactive protein is increasingly recognized inflammatory biomarker in blood (but not urine) that is used to monitor for development of cardiovascular disease. Urinary albumin, measured as an albumin/creatinine ratio, is used clinically to measure microalbuminuria, with the increased levels of this specific protein associated with elevated risk for kidney and cardiovascular diseases. Similarly, elevated isoprostane levels (oxidative damage biomarkers in blood or urine) have been reported to be independent risk markers for cardiovascular disease with statistics comparable to CRP or HDL/LDL ratio, but isoprostane measurements are typically complex and have not found wide-spread application. However, the use of antioxidant capacity has been only applied to human physiologic fluids in academic research studies, and the use of test panels incorporating multiple biomarkers have been restricted to inflammatory biomarkers or oxidative stress biomarkers, typically without inclusion of antioxidant markers, and typically including inflammatory and oxidative stress markers only in very large, expensive, broad clinical testing that include 20 or more biomarkers with comprehensive analysis or interpretation of the results referred to a physician.

The incorporation of a small number of relatively broad tests for oxidative damage and inflammation with a broad test for antioxidant activity provides, for the first time, a relatively rapid, broad, and affordable screening panel to assess an individual's wellness and susceptibility to major chronic diseases. By including information regarding their body mass index, and/or information regarding the test subject's age, lifestyle and disease history, and linking the numerical results to a database of specific interpretive narratives drawn from the scientific literature regarding the import of the data and methods (including specific diets, exercise, etc.) to improve the values relative to a person's age, the systems and methods for monitoring health herein provide an unprecedented approach to improved screening of broad populations for health and wellness, and for the feedback needed to help effect behavioral changes to improve health.

### Normalization of Urinary Biomarker Assay Measurements

As mentioned, a test strip comprising one or more biomarker assays may further comprise a normalization assay usable to adjust other determinations for urine concentration. The concentration of substances in urine can vary widely, depending on an individual's consumption of water, sweat, etc. Methods that allow for adjustment for urinary output include (a) performing studies on first morning specimens (most concentrated, but inconvenient, still variable and not always reliable), (b) collection of a 24-hour urine specimen (very reliable but very inconvenient and rarely used anymore), and (c) normalization of values to a metabolite that is excreted at a relatively constant rate or to the specific gravity of the specimen. Among the latter, creatinine is most commonly used.

The amount of creatinine excreted daily by an individual is relatively constant. Therefore, urinary creatinine levels in an individual can be used to normalize the levels of other urinary biomarkers measured by a test strip, such as levels of urinary MDA. Urinary creatinine levels in males range from about 20 to 370 mg/dL, and for females from about 20 to about 320 mg/dL. There are relatively few conditions for which the use of creatinine for normalization of the levels of substances in urine is not 100% accurate. Therefore, normalization of biomarker values to the concentration of creatinine is very common in clinical medicine, in medical research and there are several established methods for performing the assay. Therefore, all of the values related to oxidative stress, antioxidant power, and inflammation are divided by the creatinine concentration. This simple process significantly improves the reliability and reproducibility and permits the tracking of changes in an individual's wellness over time and as the result of changes in diet, lifestyle, etc.

For elite athletes, i.e. professional athletes and those engaging in serious bodybuilding or regular marathon running and training, creatinine levels may be abnormally elevated. For example, anaerobic strength training, the crux of bodybuilding, results in huge creatinine production. Further, many body builders take supplements high in creatinine. In the case of athletes, an extra normalization assay is required so that the level of creatinine measured can be normalized. Although not an assay per se, specific gravity of a urine sample provides a normalization mechanism when urinary creatinine cannot be relied upon for normalization. An individual may be provided a simple way to measure the specific gravity, (e.g. a simple hydrometer that can be submerged in the sample) and then input the value read into an app.

In various aspects, a creatinine assay for a colorimetric reagent test strips may comprise copper sulfate (CuSO4), 3,3′,5,5′-tetramethylbenzidine (TMB) and diisopropyl benzene dihydroperoxide (DBDH) dried into a test pad positioned on the strip. Creatinine in urine combines with copper sulfate to form copper-creatinine peroxidase that reacts with DBDH and releases oxygen ions that oxidize TMB causing a color change that can be measured by the assay reading device.

Since it is also known that urine absorbs light and that the color of a urine sample depends on both endogenous substances and ingested substances, such as foods and medications, a test strip of assays herein may further comprise an adjustment mechanism for adjusting the measurement of specific biomarkers to correct for color or fluorescence in the urine from irrelevant substances.

### Group-Specific Biomarker Combinations

As discussed, test strips herein are customized so that they are appropriate and optimized for particular groups of individuals. The groups as discussed may include normal adult, workplace worker (for workplace wellness compliance of employees, for example), elderly, athlete, and dieter. In each group, the system and method for health monitoring, which are adaptations of the metabolic urine test core platform, comprises a unique test strip further comprising a critical combination of biomarker assays and a normalization assay. In all groups except athlete, the normalization assay may comprise a urinary creatinine assay. In any group, the test strip may comprise a combination of biomarkers that give some indication of at least one of oxidative stress, antioxidant capacity, inflammation, and/or toxicity. In various aspects, a urinary test strip for the normal adult and the urinary test strip for workplace wellness may be the same. However, for workplace wellness, HIPPA (health insurance portability and accountability act) compliance is required. Therefore, methods of monitoring an individual's health for a person in the normal adult group versus a person in the workplace worker group may vary only in the software present on the non-transient computer-readable media of the person's PED.

TABLE 2 sets forth the critical combinations of biomarker assays for each group of individuals. In some instances, substitutions of biomarker assays are possible as indicated. In TABLE 2, an asterisk (*) indicates the creatinine assay is present for the purpose of providing a normalization mechanism for the other biomarker measurements. A double asterisk (**) indicates the creatinine assay is present as a biomarker relating to the health of an individual. In this single case of the athlete, a separate urinary normalization assay is required such that the creatinine level measured, which can be abnormally elevated in anaerobic training, can be normalized to account for variances in urine concentration.

TABLE 2
Biomarker Assay Combinations for Each Group of Individuals

| | Group of Individuals | | | | |
|---|---|---|---|---|---|
| | Normal | Workplace | | | |
| Biomarker | Adult | Worker | Elderly | Athlete | Dieter |
| Creatinine | ✓* | ✓* | ✓* | ✓** | ✓* |
| MDA | x | x | x | x | x |
| PDX | x | x | | x | x |
| CUPRAC | x | x | | x | |
| UTI | x | x | x | x | x |
| PRO | x | x | x | | x |
| Ketone | x | x | x | x | x |
| pH balance | x | x | | | x |
| Fat metabolizing | | | | x | x |
| Muscle efficiency | | | | x | |
| Glucose | | | x | | |
| Electrolytes | | | x | | x |
| Blood | | | x | | |
| Nitrite/nitrate | | | | x | |
| Bilirubin | | | x | | |
| Leukocytes | | | x | | |

As a personal health/nutrition tool available directly to the consumer, the systems and methods of health monitoring disclosed herein serve the purpose of monitoring the positive benefits of diet, rest, hydration and exercise, providing positive reinforcement for healthy lifestyle choices by the ordinary adjust, and a wealth of useful information, links to products and services, personal history tracking, online forum, and the like. Since the systems and methods herein comprise a health monitoring tool, rather than a diagnostic system, the ranges of biomarkers being assessed fall somewhere within a ‘normal’ distribution but can be tracked for trends, and intervention services are non-medical in nature, typically, smoking cessation, diets, fitness programs, etc., as offered by wellness consultants and employee health programs already. Normal adults already experiencing high cholesterol levels or obesity can benefit from even small alterations in their behavior, as motivated by the results of their own real-time health monitoring. Biomarkers levels found outside a normal range for the age group/gender warrant contacting a medical professional.

In various embodiments a system for monitoring an individual's health comprises an adaptation of the metabolic urine test core platform to a particular group of individuals. In various aspects, a system for monitoring an individual's health comprises a test strip further comprising at least one biomarker assay providing information relating to health and at least one normalization assay for normalizing the levels determined in the other biomarker assays, an assay reading device capable of reading changes to the assays upon exposure to a urine sample, and software stored on a non-transient computer-readable medium that providing instructions for normalizing the biomarker determinations and for indexing the normalized levels to lookup charts comprised of biomarker ranges deemed normal for individuals of same age and gender, and similar lifestyle. In this way, the system provides a health assessment of the individual from a urine sample.

In various aspects, malondialdehyde (MDA) is measured by an assay arranged on a test strip suitable for use with one or more of the groups of individuals. MDA is one of the major byproducts of lipid peroxidation, which occurs as a result of free radicals attacking membrane lipoproteins and polyunsaturated fatty acids. This molecule is a highly reactive electrophilic aldehyde with both mutagenic and carcinogenic properties that can covalently bond to DNA, proteins, and other cellular constituents causing modifications and damage. MDA can also enter the blood stream and travel to remote organs and tissues causing oxidative damage or can perpetuate the lipid peroxidation process by forming more reactive aldehydes. A variety of cellular enzymes exist to degrade MDA. An MDA assay herein comprises a stable dry chemistry test pad comprising dried chemical reagents or a lateral flow assay based on enzymes and/or antibodies. The MDA assay provides a color that is quantifiable by the assay reading device equipped with a colorimeter.

In various aspects, hydrogen peroxide (H₂O₂) is measured by an assay arranged on a test strip suitable for use with one or more of the groups of individuals. Hydrogen peroxide is a ubiquitous, biological signaling molecule important for various functions, including, cellular proliferation and differentiation, inflammation and the immune response, insulin signaling, neuronal and glial signaling, circadian rhythm, and the aging process. It is also a metabolic byproduct of oxidative stress and oxidative injury. H₂O₂ itself is not a strong oxidizing or reducing agent, however, when paired with metals, such as iron, a very powerful hydroxyl radical is formed that can lead to extensive damage to proteins, lipids, and DNA. As such, levels of H₂O₂ must be tightly regulated by enzymes that catalyze the breakdown of this and other reactive oxygen species (ROS). Superoxide, which is released as a byproduct of mitochondrial respiration and in reaction to pathogen invasion, is broken down into H₂O₂ by the scavenging enzyme superoxide dismutase. In turn, catalase and other peroxidase enzymes are responsible for the decomposition of H₂O₂ to water and oxygen. The normal adult range for H₂O₂ in urine is 0.0008 to about 0.0875 mg/dL. An H₂O₂ assay herein comprises a stable dry chemistry test pad comprising dried chemical reagents or a lateral flow assay based on enzymes and/or antibodies. The H₂O₂ assay provides a color (e.g. by formation of a complex between Xylenol Orange and ferric ions) that is quantifiable by the assay reading device equipped with a colorimeter. In other examples, an enzymatic method is used to generate a color reaction that is measured by multi-wavelength reflectance.

In various aspects, aggregate antioxidant capacity (AAC) is measured by an assay arranged on a test strip suitable for use with one or more of the groups of individuals. Antioxidant capacity, a measure of an organism antioxidant defense, is influenced by a wide range of factors. Studies have shown that antioxidant capacity is reduced in sedentary individuals, smokers, and that it declines with age. Decreased antioxidant capacity has also been observed in anorexia nervosa, AIDS, cardiomyopathy, diabetic neuropathy, ischemia-reperfusion injury and Crohn's disease. Uric acid, a major antioxidant in human plasma, has been demonstrated to correlate with and predict the development of obesity, hypertension and cardiovascular disease—all conditions that are associated with oxidative stress. It is therefore important to be able to quantitatively assess the total antioxidant power or capacity within biological specimens. However, given the large number of antioxidant systems employed by humans to prevent oxidative damage, along with the wide range of dietary components that have demonstrated antioxidant activity, measuring any one of these individually does not adequately assess redox status. The normal antioxidant capacity of human urine is about 3 to about 50 mg/dL. Total antioxidant capacity may be measured in an assay comprising TEAC, TRAP, FRAP, ORAC or CUPRAC.

Aggregate antioxidant capacity (AAC) assay is perhaps more reliable than TEAC, TRAP or FRAP, for example, and is adaptable to a dry chemistry test pad or lateral flow assay. AAC is more reproducible and reliable than other measurements of total antioxidant capacity. The AAC assay based on a copper redox reaction provides a color that is quantifiable by the assay reading device equipped with a colorimeter.

In various aspects, urinary trypsin inhibitor is measured by an assay arranged on a test strip suitable for use with one or more of the groups of individuals. Proteolytic enzymes, such as serine proteases, a family of enzymes that catalyze the cleavage of peptide bonds, are involved in countless biological processes, which include inflammation and the immune response. Dysregulation of serine proteases can lead to a sustained state of inflammation. Urinary trypsin inhibitors are peptides with various physiologic roles, including inhibition of the inflammatory cascade by acting as a serine protease inhibitor. Urinary trypsin inhibitors are predictive of vascular inflammation, which damages endothelial and epithelial layers and promotes vascular disease. They are normally excreted in urine and levels correlate with other markers of inflammation, such as CRP (C-reactive protein). Urinary trypsin inhibitor levels in urine are useful, both as an early marker of inflammation and an indication of inflammation-induced systemic proteolysis, and are increased in the urine of patients with congestive heart failure, vascular disease, cancer, diabetes, and kidney disease. The normal range of urinary trypsin inhibitor in human urine is ≤1.875 mg/dL. Values ≥3 mg/dL are usually indicative of bacterial infections, viral infections, and/or inflammatory disorders. A urinary trypsin inhibitor assay herein comprises a stable dry chemistry test pad comprising dried chemical reagents or a lateral flow assay based on enzymes and/or antibodies. An enzymatic method provides color inversely proportional to concentration, and the color is quantifiable by the assay reading device equipped with a colorimeter.

In various aspects, urinary proteins are measured by an assay arranged on a test strip suitable for use with one or more of the groups of individuals. When functioning normally, the protein permeability of the glomerular capillaries in the kidneys filter out large molecules from the urine, including most proteins. There is a large body of scientific literature documenting that chronic inflammation reduces the glomerular filtration rate (GFR) and increases the level of urinary protein. The impact of renal insufficiency was demonstrated in a study of over one million people showing that decreased GFR values are associated with adverse cardiovascular events and an increased risk of death. Likewise, increased urinary proteins are a marker for metabolic syndrome, diabetes, diabetic nephrology, renal disease, and cardiovascular disease. Microalbuminuria, defined as the excretion of 30 to 300 mg of albumin in urine in a 24 hour period, is an early warning for the development of metabolic syndrome, which is characterized by impaired insulin sensitivity, impaired glucose tolerance, hypertension, and dyslipidemia. Of all the co-morbidities present in metabolic syndrome, microalbuminuria carries the strongest risk for cardiovascular death. Among patients with Type-I and Type-II diabetes, microalbuminuria is a specific marker for the development of diabetic nephrology and cardiovascular disease. The average normal concentration of protein in urine is 2-8 mg/dL or 40-150 mg excretion over a 24-hour period. Clinical proteinuria is characterized by >500 mg of protein per day. A protein assay herein comprises a stable dry chemistry test pad comprising dried chemical reagents comprising acid-base indictors that change color in the presence of protein, wherein the color is quantifiable by the assay reading device equipped with a colorimeter.

In various aspects, urinary ketones are measured by an assay arranged on a test strip suitable for use with one or more of the groups of individuals. Ketone bodies are intermediate products that occur naturally during fatty acid metabolism. Hepatic cell mitochondria break down acetyl-CoA into three types of ketones: acetone, acetoacetic acid, and β-hydroxybutyric acid. Acetone, a volatile waste product, is mainly excreted by lungs. Both acetoacetic acid and β-hydroxybutyric acid are released into circulation where they can be detected in blood and urine. Under normal conditions, no ketones are present in urine. The formation of ketones may dramatically increase during physiological stressors where carbohydrate stores are depleted (during fasting/starvation diets, strenuous exercise, pregnancy) and can be indicative of diabetes, diabetic ketoacidosis, or other metabolic disorders. Significantly higher levels of urinary ketones have been associated with increasing severity for heart failure patients. Since ketones are detectable in urine prior to measurable levels being present in the blood, they are a useful early risk indicator. Typically, little or no ketones are excreted in the urine. Low levels of 3-15 mg are considered to be in the normal range. In ketoacidosis, starvation or with other abnormalities of carbohydrate or lipid metabolism, ketones may appear in urine at levels of 10 mg/dL or higher before serum ketone levels are elevated. A ketone assay herein comprises a stable dry chemistry test pad comprising dried chemical reagents based on a sodium nitroprusside reaction, where the purple color developed is quantifiable by the assay reading device equipped with a colorimeter.

In various aspects, pH of the urine sample is measured by an assay arranged on a test strip suitable for use with one or more of the groups of individuals. Urinary pH is an indication of kidney function, specifically, the kidneys' reabsorption and secretion of sodium, potassium, calcium, ammonia, and other salts to maintain a balance of acids and bases produced by normal metabolic processes. The pH level indicates the amount of acid (or hydrogen ions) in urine. In general, the urine pH is consistent with the serum pH, but abnormal levels may indicate a kidney or urinary tract disorder. The urine pH can also be related to diet and other factors. Highly acidic urine has been shown in diabetes mellitus. The normal pH of human urine is about 6, however pH may range from about 4.6 to about 8.0. A pH assay herein comprises a stable dry chemistry test pad comprising dried acid-base indictors that change color based on pH , wherein the color is quantifiable by the assay reading device equipped with a colorimeter.

### Computer Processing

In various embodiments, a method for monitoring an individual's health is computer implemented. Further, a system for monitoring an individual's health comprises a computer with a non-transient computer-readable medium, such as a CPU, on which program instructions (i.e., algorithms, or “software”) are written for carrying out the method of monitoring an individual's health. In other variations, software is stored on the Cloud or a remote server, such as a mainframe computer managed by a healthcare provider. In various aspects, a computer herein comprises a PED. In various embodiments, a computer may comprise a desktop computer, a laptop, a tablet or a smartphone. The PED comprises both hardware and software along with the necessary connections (hardwire/USB or wireless) to the device that reads the results of the various biomarker assays, thus enabling the method. Data may be manually entered into the PED, such as through a keyboard/mouse or a touchscreen.

In various embodiments, a software app is downloaded onto a PED, such as a smartphone, and the smartphone is connected via a port or by Bluetooth to the assay reading device. A downloaded app may appear as an icon on the display screen of a smartphone. The software app comprises code written to convert the numerical values obtained from the device into a health assessment, by normalizing and indexing the biomarker levels measured and taking into account personal information of the individual desiring the health assessment.

In various embodiments, the non-transient computer-readable media contains program instructions that process all the biomarker data obtained from the assay reading device. In various aspects, the software includes an algorithm that uses the numerical result obtained from one normalization assay to normalize the numerical result obtained from another biomarker assay. In the simplest example, the software includes a division algorithm and the result of a biomarker assay is divided by the result of the normalization assay, such as a urinary creatinine assay. In other examples, the normalized biomarker level in the individual's urine is compared to a table of normal urinary biomarker levels based on age, gender, and if female, if pregnant. In this way the software first normalizes the biomarker result with the normalization assay, and then compares that normalized biomarker value to a chart of normal values to ascertain if the individual is healthy or not. Further the program instructions may provide a report to the individual regarding their health, such as in the form of a display on a smartphone.

The personal information entered into the app may include an individual's gender, age, height, and weight such that the written code may calculate an individual's body mass index (BMI), as well as information regarding the individual's lifestyle (e.g. tobacco and/or alcohol use) and other factors such as preexisting medical conditions (e.g. diabetes). Other personal information may include the individual's choice for one or more of the groups identified above (normal adult, etc.). Since it is well documented that antioxidant activity declines with age and that oxidative stress tends to increase with age, age-related normalization can also be performed on the biomarker assay results. The BMI can be used in comparisons with the results of the various biomarker assay tests on the test strip, i.e. BMI versus oxidative damage, BMI versus antioxidant power, BMI versus oxidative stress (OS) status, BMI versus inflammation, and so forth. The BMI can be compared to the test results in order to determine risk for diseases.

### Other Considerations and Embodiments

A method for monitoring an individual's health is performed as follows. The individual in need of a health assessment or participating in a health monitoring program supplies their own urine sample. The test strip appropriate for that person's group is wetted with the urine sample. Once wetted with the urine sample, the various biomarker assays performing prescribed tests for at least one biomarker relating to health and at least one normalization assay. In various embodiments, biomarker assays measure at least one of oxidative stress, antioxidant capacity, inflammation and/or toxicity. The biomarker results are normalized to correct for the relative concentration of the urine sample, and normalized levels are compared to normal ranges for individuals of the same gender and age and of similar lifestyles.

A sample for analysis by the test strip is easily obtained from an individual's urine. The sample can be obtained by a cup for use with a dipstick style test strip that is placed in the urine.

Analysis of one or more biomarkers, preferably two each for oxidative stress and inflammation to improve reliability and reduce errors associated with confounding factors that can influence specific biomarkers, for each of the three conditions is performed as specified above by the test strip. When a dipstick is used as the test strip, detecting a color change in the dipstick by the assay reading device can indicate the measurement of specific biomarkers in each test pad of the test strip. Each test can change the amount of colored light reflected from one of the components of the dipstick. For a negative result (i.e. the presence of a biomarker is not detected), the strip can remain its original color, or it can change to a specific color. For a positive result (i.e. the presence of a biomarker is detected), the strip can change to a distinctively different color than the negative result. One example is the strip turning blue for a negative result and pink for a positive result. In preferred embodiments, the results are non-qualitative (color versus lack of color) but vary in degree corresponding to the level of the biomarker present. For example, an intense color can indicate the presence of high levels of the specified biomarker, and a muted color can indicate the presence of low levels of the biomarker.

Subsequently, the dipstick or other dry chemistry test strip can be inserted into an assay reading device that quantifies the reflected color for each test pad and a quantitative value can be transmitted to the CPU for analysis. In this method, the amount of each biomarker present can be determined to provide further information as to the health of the user. In other words, lower or higher levels of biomarkers, and not just their presence, can be relevant to the state of health. Alternatively, the individual makes a visual evaluation of the colors or other changes to the assays on a test strip and manually enters the information on his/her smartphone.

The assay reading device can include or be coupled to the PED having software instructions written on a non-transient computer-readable media that is capable of performing analysis using the data thus obtained from the assay reading device. The written code computes values of each of the biomarkers in the tests, performs normalization as described above, as well as compute relationships of the test results with each other, the test results with BMI described above or, after calculating oxidative stress and antioxidant capacity, the ratio of both can be calculated to determine OS (oxidative stress) status and this value can be compared with BMI or inflammation. The software may also contain instructions to search a database for facts relating high or low levels of specific biomarkers to disease risks, and can include facts derived from scientific literature that provide suggestions for lifestyle changes, or suggestions for further testing based on the test results, and combinations thereof.

The presence of biomarkers for health can then be indicated to the user. The written instructions further include an output to display the results in a meaningful way to an individual or health care practitioner. The display can be on the screen of a smartphone or on another device, and may be printed off a printer. Alternatively, the software can also send the results over wireless signals or wires to a PDA, smart phone, or a remote computer for print out or display. The results can be incorporated into a report on an individual's wellness that includes, but is not limited to, the results of the tests, comparison to the values and ratios computed to normal ranges that have previously been established for normal healthy men and women of different ages, ethnicities (if relevant) and/or other relevant parameters. Such a report can also incorporate historical data for an individual subject that was obtained using the same method(s). The report can further show the information from the database described above.

The systems and methods of health monitoring disclosed herein find use in wellness programs administered by insurance companies or large insurers, by employers, by clinicians, nutritionists, wellness consultants, and others as well as fitness and training programs administered by sports organizations or the military. The preferred use of the systems and methods herein is a point of testing health and wellness assessment, which can be performed in a doctor's office, by a health care practitioner or an insurance agent after suitable training. The systems and methods can also be used by individuals to monitor their health in their own home.

The systems and methods of the present disclosure including multiple biomarkers in various combinations provide better results than individual assays for the various biomarkers discussed herein. Tests for inflammation, oxidative stress, antioxidant activity have been studied independently and in controlled studies for large numbers of subjects, each has been associated with disease and/or disease risk. Oxidative stress and inflammation often increase or decrease together, and it is known that certain transcription factors are involved. For example, oxidative stress turns on the expression of some genes encoding some inflammatory proteins and vice versa. However, each of the specific tests for oxidative stress and inflammation biomarkers is subject to some confounding factors as discussed above. Hence, elevated urinary protein can result from strenuous exercise or athletic training and not inflammation (although overexertion can cause inflammation); NOx may be falsely and transiently elevated by eating some hot dogs; MDA will transiently increase following athletic training—but endogenous sources for antioxidant activity are increased by exercise. By comparison to one's lipid profile, it is much more informative to measure a panel of biomarkers, just as one's cholesterol or HDL level alone does not provide as complete and accurate a picture. There are multiple endogenous and exogenous variable that can confound any of the assays in TABLE 1. By employing a test strip with more than one but a manageable number of biomarker assays, one can improve the reliability of the overall method versus one test or even one test for each condition.

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

As used herein, “satisfy”, “meet”, “match”, “associated with” or similar phrases may include an identical match, a partial match, meeting certain criteria, matching a subset of data, a correlation, satisfying certain criteria, a correspondence, an association, an algorithmic relationship and/or the like.

Terms and phrases similar to “associate” and/or “associating” may include tagging, flagging, correlating, using a look-up table or any other method or system for indicating or creating a relationship between elements, such as, for example, (i) an account and (ii) a healthcare asset and/or digital channel. Moreover, the associating may occur at any point, in response to any suitable action, event, or period of time. The associating may occur at pre-determined intervals, periodic, randomly, once, more than once, or in response to a suitable request or action. Any of the information may be distributed and/or accessed via a software enabled link, wherein the link may be sent via an email, text, post, social network input and/or any other method known in the art.

### EXAMPLES

### Example 1

### Colorimetric Assay Results Flow Chart-From Sample to Display

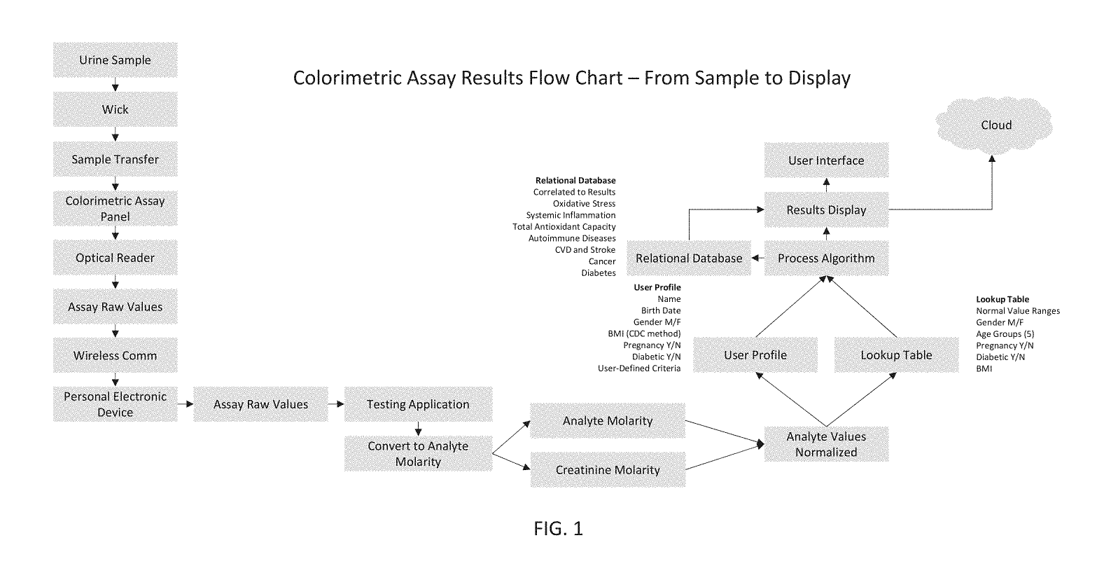

A urine sample is obtained through a wick. The sample is transferred to a colorimetric assay panel. An optical reader assays raw values. The raw values are then wirelessly communicated to a personal electronic device. A testing application converts the raw values to molar values, which includes analyte molarity and creatinine molarity. Analyte values are normalized to lookup tables that comprise biomarker ranges that are considered normal for people of the same age and gender of the user. In addition, analyte values are normalized as they relate to normal biomarker ranges related to other facts about the user, such as whether the person is pregnant, whether the person is a diabetic, and the BMI of the individual. A process algorithm assesses those results in terms of a relational database to determine if the individual has any of a number of physiological conditions such as oxidative stress, systemic inflammation, total antioxidant capacity, autoimmune diseases, cardiovascular disease, cancer, and diabetes. The results are then displayed on a user interface, which can be stored in a cloud. An example of a flow chart of this process is shown in FIG. 1.

### Example 2

### User Interface

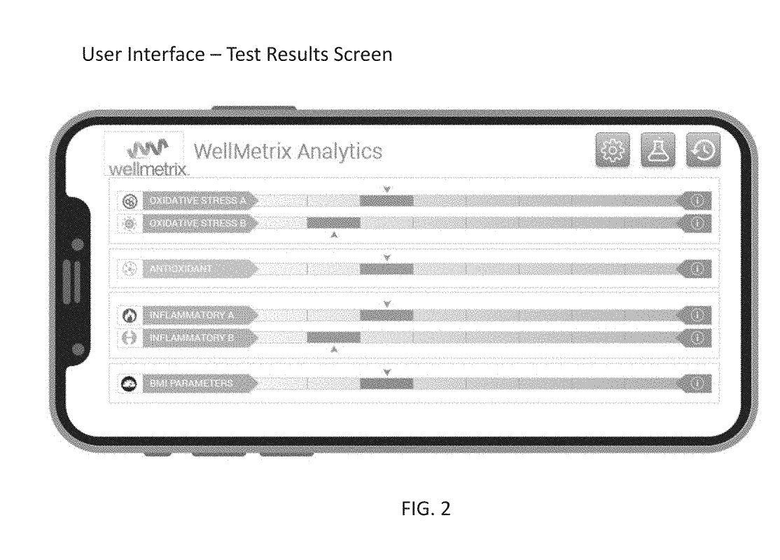

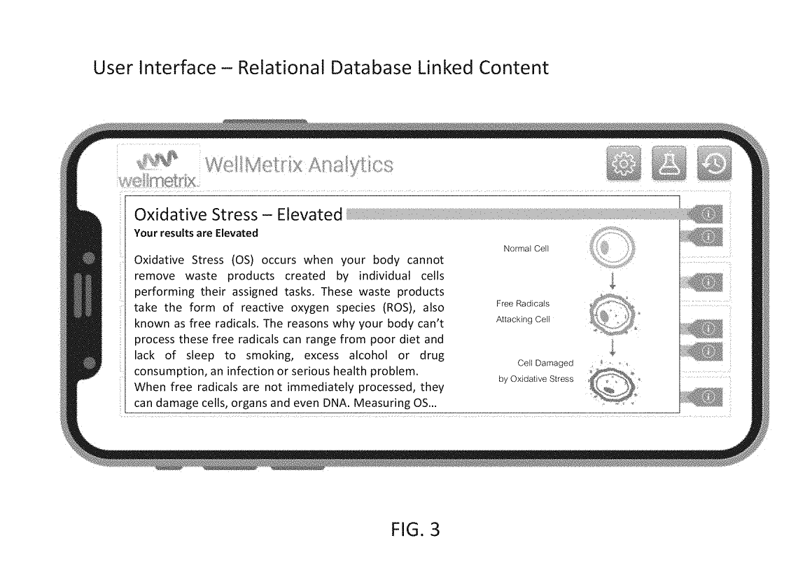

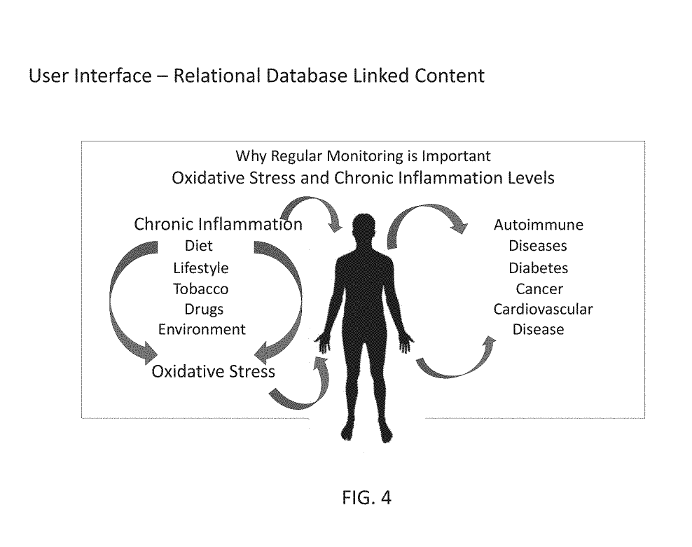

The user interface can display test results and relational database linked content. An example of a result displayed on a user interface is shown in FIG. 2. In FIG. 2, normalized biomarkers related to BMI are compared against oxidative stress, antioxidants, and inflammation. Examples of relational database linked content is shown in FIG. 3 and FIG. 4. FIG. 3 provides an example of relational database linked content that describes how the users oxidative stress is elevated. FIG. 4 shows an example of a user interface of relational database linked content describing why regular monitoring is important. In particular, it shows how chronic inflammation that derives from diet, lifestyle, tobacco use, drug use or the environment as well as oxidative stress can result in autoimmune diseases, diabetes, cancer, and cardiovascular disease

### Example 3

### System Components

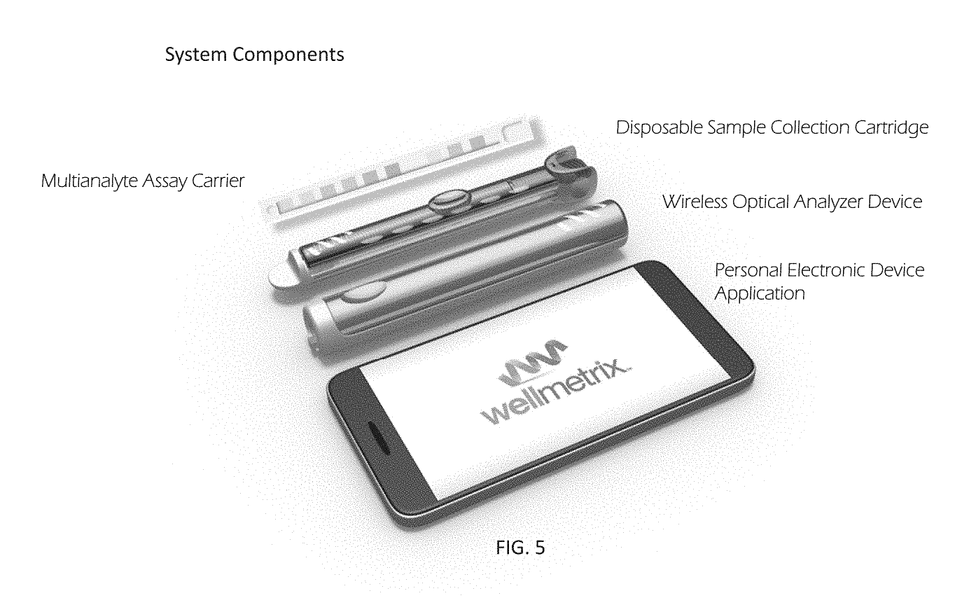

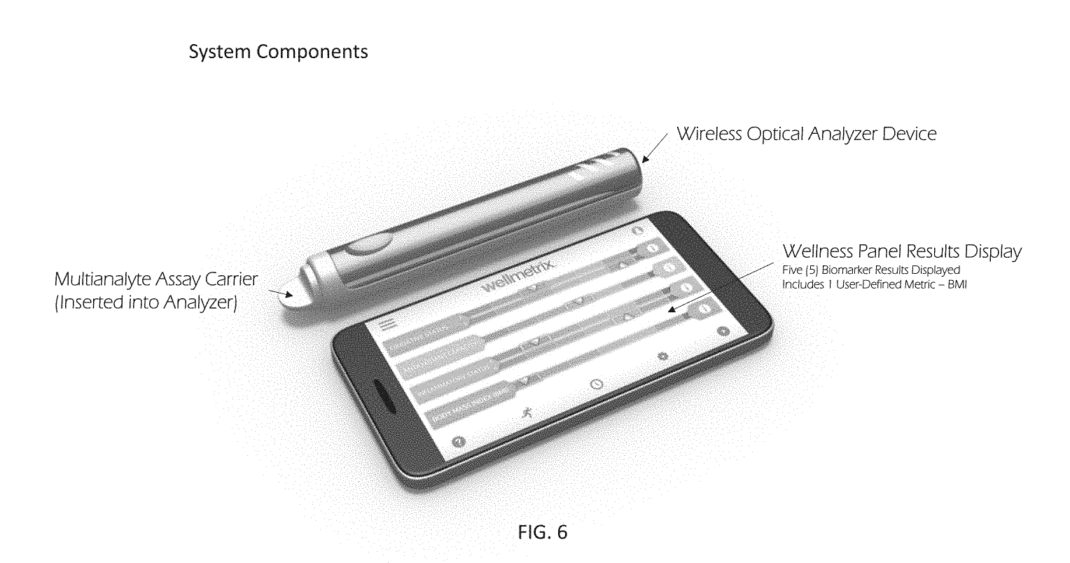

The system can have various system components. FIG. 5 provides one embodiment of the system components, which comprise a multianalyte assay carrier, a disposable sample collection cartridge, a wireless optical analyzer device, and a personal electronic device application. FIG. 6 provides another embodiment of the system components, which comprise a multianalyte assay carrier inserted into an analyzer, a wireless optical analyzer device, and a wellness panel results display.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus

1. A metabolic urine core test platform comprising: a model; a physiologic fluid; a test panel comprising a biomarker assay; an instrument; and a computer, the computer further comprising program instructions written on a non-transient computer-readable media for performing a method of monitoring at least one biomarker.

2. The metabolic urine core test platform of claim 1, wherein the model comprises human individuals classified into groups.

3. The metabolic urine core test platform of claim 2, wherein the groups are selected from the group consisting of normal adult, a workplace worker, an elderly person, an athlete and a dieter.

4. The metabolic urine core test platform of claim 2, wherein the physiologic fluid comprises urine and the biomarker assay comprises a urinary test strip, the test strip further comprising a urinary normalization assay arranged on the same strip with the biomarker assay, wherein the assays comprise chemical, enzymatic or immunochemical assays.

5. The metabolic urine core test platform of claim 4, wherein the test strip comprises a critical combination of biomarker assays in addition to the urinary normalization assay, wherein the critical combination of biomarker assays is determined by selection of the group of individuals.

6. The metabolic urine core test platform of claim 5, wherein the critical combination of biomarker assays relate to at least one of oxidative stress, antioxidant capacity, inflammation, and toxicity of the individual.

7. The metabolic urine core test platform of claim 4, wherein the instrument comprises a portable or handheld assay reading device comprising a colorimeter for reading color on the assays.

8. The metabolic urine core test platform of claim 5, wherein the computer comprises a PED and the program instructions written on the non-transient computer-readable media comprise an app downloadable to the non-transient computer-readable media of the PED.

9. The metabolic urine core test platform of claim 8, wherein the instrument electronically connects to the PED by hardwire or Bluetooth, to enable data transfer from the instrument to the non-transient computer-readable media of the PED.

10. The metabolic urine core test platform of claim 1, wherein the method of monitoring comprises:
obtaining the physiologic fluid sample from the individual;
wetting the test panel with the physiologic fluid sample to create a wetting test panel;
inserting the wetted test panel into the instrument;
transferring data from the instrument to the non-transient computer-readable media of the computer; and
analyzing the data to provide an assessment of the individual's health.

11. A system for monitoring health of an individual comprising:
a urinary test strip comprising a combination of biomarker assays and a urinary normalization assay, the assays positioned and spaced apart on the strip;
an assay reading device comprising a colorimeter, the device configured for physical insertion of the urinary test strip therein, the colorimeter configured for obtaining colorimetric data from the assays; and
a computer comprising program instructions written on a non-transient computer-readable media for performing transfer of the data from the assay reading device to the non-transient computer-readable media of the computer and analysis of the data to provide the health of the individual.

12. The system of claim 11, wherein the individual is selected from the group consisting of a normal adult, a workplace worker, an elderly person, an athlete, and a dieter.

13. The system of claim 11, wherein the biomarker assays and the urinary normalization assay are selected from dry reagent test pads and/or lateral flow immunoassays.

14. The system of claim 11, wherein the urinary normalization assay comprises a urinary creatinine assay.

15. The system of claim 11, wherein the computer comprises a PED.

16. The system of claim 12, wherein the group comprises the normal adult, the combination of biomarker assays comprises MDA, PDX, CUPRAC, UTI, PRO, Ketone, pH, and the urinary normalization assay comprises a urinary creatinine assay.

17. The system of claim 12, wherein the group comprises the workplace worker, the combination of biomarker assays comprises MDA, PDX, CUPRAC, UTI, PRO, Ketone, pH, and the urinary normalization assay comprises a urinary creatinine assay.

18. The system of claim 12, wherein the group comprises the elderly, the combination of biomarker assays comprises MDA, UTI, PRO, Glucose, Electrolytes, Blood, Bilirubin, Leukocytes, and the urinary normalization assay comprises a urinary creatinine assay.

19. The system of claim 12, wherein the group comprises the athlete, the combination of biomarker assays comprises a urinary creatinine assay, MDA, PDX, CUPRAC, UTI, Ketone, Fat metabolizing, Muscle efficiency, Nitrite/Nitrate, and the urinary normalization assay comprises a specific gravity measurement.

20. The system of claim 12, wherein the group comprises the dieter, the combination of biomarker assays comprises MDA, PDX, UTI, PRO, Ketone, pH, Fat metabolizing, Electrolytes, and the urinary normalization assay comprises a urinary creatinine assay.

21. A method of monitoring an individual's health, the method comprising:
obtaining a urine sample from the individual;
wetting a urinary test strip with the urine sample;
inserting the wetted urinary test strip into an instrument in communication with a computer and configured to read data from the wetted urinary test strip when inserted therein;
reading the data from the wetted urinary test strip;
transferring the data from the instrument to a non-transitory computer-readable media of the computer; and
analyzing the data to obtain the individual's health,
wherein program instructions for performing the method are written on the non-transitory computer-readable media.

22. The method of claim 21, wherein the urinary test strip further comprises a combination of biomarker assays and one urinary normalization assay.

23. The method of claim 22, wherein the individual is classified into at least one group based on age and/or lifestyle, and wherein the combination of biomarker assays is determined by the nature of the group.

24. The method of claim 23, wherein the group is selected from normal adult, workplace worker, elderly, athlete and dieter.

25. The method of claim 24, wherein the instrument comprises an assay reading device further comprising colorimetry capability for reading color from the combination of biomarker assays and one urinary normalization assay.

26. The method of claim 24, wherein the group comprises the normal adult, the combination of biomarker assays comprises MDA, PDX, CUPRAC, UTI, PRO, Ketone, pH, and the urinary normalization assay comprises a urinary creatinine assay.

27. The method of claim 24, wherein the group comprises the workplace worker, the combination of biomarker assays comprises MDA, PDX, CUPRAC, UTI, PRO, Ketone, pH, and the urinary normalization assay comprises a urinary creatinine assay.

28. The method of claim 24, wherein the group comprises the elderly, the combination of biomarker assays comprises MDA, UTI, PRO, Glucose, Electrolytes, Blood, Bilirubin, Leukocytes, and the urinary normalization assay comprises a urinary creatinine assay.

29. The method of claim 24, wherein the group comprises the athlete, the combination of biomarker assays comprises MDA, PDX, CUPRAC, UTI, Ketone, Fat metabolizing, Muscle efficiency, Nitrite/Nitrate, and the urinary normalization assay comprises a specific gravity measurement.

30. The method of claim 24, wherein the group comprises the dieter, the combination of biomarker assays comprises MDA, PDX, UTI, PRO, Ketone, pH, Fat metabolizing, Electrolytes, and the urinary normalization assay comprises a urinary creatinine assay.
